(19) United States
(12) Patent Application Publication
(10) Pub. No.: US 2008/0014155 A1
(43) Pub. Date: Jan. 17, 2008

(54) CLEAR SUNSCREEN COMPOSITIOIN

(76) Inventor: CHRISTOPHER MARRS, Rancho Palos Verdes, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/755,842

(22) Filed: May 31, 2007

Related U.S. Application Data
(60) Provisional application No. 60/803,565, filed on May 31, 2006.

(51) Int. Cl. A61K 8/00; A61Q 19/00

(52) U.S. Cl. 424/59

(57) ABSTRACT

The present invention relates clear sunscreen compositions. The compositions include an organic sunscreen and an alcohol. In certain embodiments, the compositions include a rheology modifier such as a non-ionic polymer and an anti-pilling agent such as a cationic polymer.

### FIELD OF THE INVENTION

The present invention relates to sunscreen compositions and, more particularly, to clear compositions including an organic sunscreen and an as alcohol.

### BACKGROUND OF THE INVENTION

The prolonged exposure to UV radiation, such as from the sun, can lead to the formation of light dermatoses and erythemas, as well as increase the risk of skin cancers, such as melanoma, and accelerate skin aging, such as loss of skin elasticity and wrinkling. Light having wavelengths in both the UV-A range (from about 320 to 400 mn) and the UV-B range (from about 280 to about 320 nm) can cause such skin damage, and, thus, sunscreen compositions should preferably comprise both UV-A and UV-B absorbers/reflectors (UV sunscreens). Furthermore, in order to provide a pleasant experience for the sunscreen user, it is also desirable for the sunscreen composition to spread easily across the skin and not create a sensation of oiliness.

Sunscreen compositions often include one or more organic sunscreens to provide broad spectrum (UV-A and UV-B) protection from ultraviolet light. However, the high concentrations of organic sunscreens required to provide protection often create a user perception of oiliness. In order to reduce this undesirable perception, formulators often emulsify the organic sunscreen in a water exterior phase (forming an oil in water emulsion). However, these compositions are often still rather oily in feel.

Another approach to reduce oiliness is to formulate the organic sunscreen in a base that includes alcohol. These systems can provide a reduced perception of oiliness and a pleasant “clear” visual appearance. Unfortunately, the alcohol-containing sunscreen compositions of the prior art suffer from considerable drawbacks, particularly poor phase and viscosity stability (e.g., at elevated temperature), limited ultraviolet protection, and a pronounced tendency to “pill” (form unaesthetic aggregations or clumps on the skin). The present invention relates to the finding of an unexpected, advantageous combination of high clarity and excellent phase and viscosity stability in a composition including an organic sunscreen and an alcohol.

### SUMMARY OF THE INVENTION

The present invention provides personal care compositions that overcome the disadvantages of the prior art. In particular, applicants have discovered compositions including an organic sunscreen and an alcohol. The composition has the unique and desirable combination of high clarity as well as one or both of phase stability and/or viscosity stability.

According to one aspect, the present invention provides a personal care composition that includes an organic sunscreen, an alcohol, and an optional rheology modifier, wherein the composition has a % transmittance_{600 nm}, greater than about 25% such as greater than about 50%, and has a syneresis stability_{40° C.} of at least about 28 days, such as at least about 56 days.

According to another aspect, the present invention provides a personal care composition that includes an organic sunscreen, an alcohol, and an optional rheology modifier, wherein the composition has % transmittance_{600 nm}, greater than about 25% such as greater than about 50%, and has a viscosity drop_{40° C., 28 days} of less than about 20%.

According to another aspect, the present invention provides a personal care composition that includes an organic sunscreen, an alcohol, a rheology-modifier such as a cellulose polymer, an anti-pilling agent such as a cationic polymer, and an optional absorbent particulate. The cellulose polymer and the cationic polymer are preferably soluble in the alcohol.

According to another aspect, the present invention provides a personal care composition that includes from about 3% to about 40%, preferably from about 5% to about 35%, more preferably from about 20% to about 30% of one or more organic sunscreens, an alcohol, a rheology-modifier such as a cellulose polymer, and an anti-pilling agent such as a cationic polymer, and less than about 10% water, such as less than about 1% water. The weight percent of alcohol may be from about 15% to about 70%, such as from about 20% to about 50%. The cellulose polymer and the cationic polymer may be present in a weight ratio that is from about 1:5 to about 5:1, such as from about 1;3 to about 3:1. The cellulose polymer and the cationic polymer are preferably soluble in the alcohol.

In yet another aspect of the invention, applicants have provided a method of treating the skin, the method including applying to the skin a composition as described above.

In yet another aspect of the invention, applicants have provided a method of protecting the skin from the damaging effects of ultraviolet radiation, the method including applying to the skin a composition as described above.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. All concentrations provided are concentrations by weight, unless otherwise specifically stated.

Compositions of the present invention include at least one organic sunscreen. What is meant by a “sunscreen” is a compound that absorbs, reflects, or scatters radiation in the CV range (280 nm-400 nm). By “organic sunscreen” it is meant any sunscreen composed predominantly of some combination of atoms of carbon, hydrogen, oxygen and/or nitrogen. The organic sunscreen may absorb in all or part of the ultraviolet spectrum and may be oil-soluble or water soluble. Suitable organic sunscreens include, for example:
3-Benzylidene camphor, specifically 3-benzylidene norcampher and derivatives thereof, e.g. 3-(4-methylbenzylidene)campher;
4-Aminobenzoic acid derivatives, specifically 4-(dimethylamino)benzoic acid-2-ethylhexyl esters, 4-(dimethylamino)benzoic acid-2-octyl esters and 4-(dimethylamino)benzoic acid amylesters;
Esters of cinnamonic acid, in particular 4-methoxycinnamonic acid-2-ethylhexylester, 4-methoxycinnamonic acid propylester, 4-methoxycinnamonic acid isoamyl ester, 2-cyano-3,3-phenylcinnamonic acid-2-ethylhexyl ester (octocrylene);
Esters of salicylic acid, i.e., salicylic acid-2-ethylhexylester, salicylic acid-4-isopropylbenzyl ester, salicylic acid homomenthyl ester;
Derivatives of benzophenones, in particular 2-hydroxy-4-methoxybenzophenone, 2-hydroxy-4-methoxy-4′-methylbenzophenone, 2,2′-dihydroxy-4-methoxybenzophenone;
Esters of benzalmalonic acid, in particular 4-methoxybenzmalonic acid di-2-ethylhexyl ester;
Triazine derivatives, for example 2,4,6-trianilino-(p-carbo-2′-ethyl-1′-hexyloxy)-1,3,5-triazine and octyltriazone; or benzoic acid, 4,4′-[[6-[[[(1,1-dimethylethyl)amino]carbonyl]phenyl]amino]-1,3,5-triazine-2,4-diyl]diimino]bis-, bis(2-ethylhexyl) ester (UVASORB HEB);
Propane-1,3-diones, for example, 1-(4-tert.butylphenyl)-3-(4′-methoxyphenyl)propane-1,3-dione;
Ketotricyclo(5.2.1.0)decane derivatives
2-Phenylbenzimidazol-5-sulfonic acid and its alkali-, alkaline earth-, ammonium-, alkylammonium-, alkanolammonium- and glucammonium salts;
Sulfonic acid derivatives of benzophenones, in particular 2-hydroxy-4-methoxybenzophenone-5-sulfonic acid and its salts;
Sulfonic acid derivatives of 3-benzylidene campher, e.g. 4-(2-oxo-3-bornylidene methyl)benzolsulfonic acid and 2-methyl-5-(2-oxo-3-bornylidene)sulfonic acid and its salts
benzoylmethane, for example, 1-(4′-tert.butylphenyl)-3-(4′-methoxyphenyl)propane-1,3-dione, 4-tert.-butyl-4′-methoxydibenzoylmethane (PARSOL 1789), 1-phenyl-3-(4′-isopropylphenyl)-propane-1,3-dione, derivatives of benzoic acid 2-(4-diethylamino-2-hydroxybenzoyl)-benzoic acid hexylester (UVINUL A+), or 1H-benzimidazole-4,6-disulfonic acid, 2,2′-(1,4-phenylene)bis-, disodium salt (NEO HELOPAN AP)
benzotriazoles, such as the benzotriazole derivative known as 2,2′-methylene-bis-(6-(2H-benzotriazole-2-yl)-4-(1,1,3,3-tetramethylbutyl)-phenol) [INCI: Bisoctyltriazol], which is commercially available under the tradename TINOSORB M from CIBA Chemicals. Another useful benzotriazole derivative is 2-(2H-benzotriazole-2-yl)-4-methyl-6-[2-methyl-3-[1,3,3,3-tetramethyl-1-[(trimethylsilyl)oxy]disiloxanyl]propyl]-phenol (CAS-No.: 155633-54-8) also identified by the INCI name drometrizole trisiloxane and available from Chimex under the tradename MEXORYL XL.
Sulfonated UV fitters such as 3,3′-(1,4- phenylenedimethylene)bis(7,7-dimethyl-2-oxo-bicyclo-[2.2.1]hept-1-yl methanesulfonic acid, and its sodium, potassium, or its triethanolammonium salts, and the sulfonic acid itself identified by the INCI name terephthalidene dicamphor sulfonic acid (CAS No. 90457-82-2), which is available, for example, under the trade name MEXORYL SX from Chimex.
Symmetrically substituted triazine derivatives. Of particular interest is 2,4-bis-{[4-(2-ethyl-hexyloxy)-2-hydroxy]-phenyl}-6-(4-methoxyphenyl)-1,3,5-triazine (INCI: anisotriazine) that is commercially available under the tradename TINO SORB S from CIBA Chemicals.

Composition of the present invention generally includes a safe and effective amount of organic sunscreen. Individually, each organic sunscreen may be present in any suitable concentration known to the art of sunscreen formulation, such as from about 0% to about 20%, preferably from about 0.1% to about 15%, more preferably from about 0.2% to about 15%. The total amount of organic sunscreen may be, for example, from about 0.1% to about 50%. Surprisingly, relatively large amounts of organic sunscreen can be incorporated into the composition. As such, in one preferred embodiment of the invention, the composition includes from about 3% to about 40%, preferably from about 5% to about 35%, more preferably from about 20% to about 30%.

Compositions of the present invention further include an alcohol that serves as a diluent for the organic sunscreen and promotes spreadability thereof across the skin, as well as promotes transparency of the composition. The term “alcohol” includes lower (C1-C6) “monohydric” alcohols, particularly alcohols that are more volatile than water, preferably ethanol or isopropanol; the term alcohol also encompasses glycols such as propylene glycol, butylene glycol, hexylene glycol; as well as polyhydric alcohols that are liquids at room temperature and pressure such as glycerol. The alcohol preferably has a molecular weight of less than about 150, preferably less than about 120, more preferably less than about 100, and most preferably between about 40 and about 80. In one particularly preferred embodiment, the composition includes ethanol.

The alcohol(s) may be present in a concentration that is from about 10% to about 90%, preferably from about 15% to about 70%, more preferably from about 20% to about 50%, and most preferably from about 25% to about 40%.

Compositions of the present invention preferably include a rheology modifier that serves to provide pleasant rheological properties, e.g., spreadability on the skin upon application of shear, resistance to “dripping” if momentarily left on a vertical skin surface, etc.

A variety of rheology modifiers are suitable for compositions of the present invention. Particularly suitable rheology modifiers are polymers that (1) form clear (preferably transparent) and stable solutions or dispersions when placed in the alcohol used in the composition such as ethanol and (2) are capable of providing a viscosity of at least about 10,000 cps (measured using a Brookfield viscometer, T spindle at 5 RPM), and preferably at least about 20,000 cps when placed therein. It is further desirable that the polymer be capable of providing a shear thinning gel (viscosity decreases with shear rate) when placed in the alcohol. Suitable rheology modifiers include, for example, synthetic or natural polymers. Certain synthetic polymers may also be utilized as the rheology modifier, providing that they form a clear, preferably transparent composition in an alcoholic system. As such, it is believed that certain polymers derived from ethylenically unsaturated monomers may be utilized. In one embodiment of the invention, the rheology modifier is a polysaccharide or derivative thereof. In one particularly notable embodiment, the rheology modifier is a non-ionic polymer, such as a cellulose polymer that has been modified to confer solubility in the alcohol used in the composition of the present invention. By “soluble in the alcohol used in the composition,” it is meant that at least 2 weight percent of the rheology modifier can form a homogeneous and clear solution when placed alone in the alcohols and agitated for a period of about 60 minutes or less. Examples of suitable alcohol-soluble cellulose polymers are those that include hydroxyl functional groups. Particular examples include hydropropyl cellulose and. One particular notable non-ionic, modified cellulose polymer is a hydroxypropyl cellulose having a weight average molecular weight from about 500,000 to about 1,200,000, such as KLUCEL MF (MW=850,000) commercially available from Hercules, Inc. of Wilmington, Del.

The rheology modifier may be present in the composition in a concentration that is from about 0. 1% to about 10%, preferably from about 0.25% to about 5%, and most preferably from about 0.5% to about 4%.

Compositions of the present invention preferably further include an anti-pilling agent. The anti-pilling agent reduces “pilling,” the formation of unaesthetic aggregations or clumps on the skin. Without wishing to be bound by theory, it is believed that the anti-pilling agent interacts with the rheology modifier or other components of the formulation to reduce the tendency of pilling as the compositions is rubbed into the skin.

The anti-pilling agent is preferably soluble in the alcohol used in the composition. In one embodiment of the invention, the anti-pilling agent is a cationic material, such as, for example, a cationic surfactant or, in a preferred embodiment, a cationic polymer. Particularly suitable are cationic materials that are soluble in the alcohol present in the composition. In one embodiment, the cationic material has a molecular weight of at least about 500, such as at least about 1000.

Examples cationic polymers include cationic polysaccharides such as naturally occurring polysaccharides that have been derivatized to create cationic character, e.g. quaternization with various quaternary amine compounds containing reactive chloride or epoxide sites. Example of cationic polysaccharides that may be suitable include, but are not restricted to cationic guar, hydrophobically modified cationic guar, cationic hydroxypropyl guar, cationic hydrophobically modified hydroxypropyl guar, cationic hydroxyethyl guar, cationic hydrophobically modified hydroxyethyl guar, cationic hydroxyethyl cellulose and cationic hydrophobically modified hydroxyethyl cellulose.

Other suitable cationic polymers include synthetic cationic polymers, such as such as may be derived from ethylenically unsaturated monomers. One suitable cross-linked cationic acrylic polymer 2-(Trimethylammoniuo)ethyl methacrylate chloride, commercially known as Polquaternium-37 and available as Synthalen CR from 3V Sigma of Italy.

The anti-pilling agent may be present in the composition in a concentration that is from about 0.1% to about 10%, preferably from about 0.25% to about 5% and most preferably from about 0.5% to about 4%.

In order to optimize low-pilling with building of a sufficient level of viscosity, as well as stability of the composition, according to one embodiment of the invention, the rheology-modifier and the anti-pilling agent are present in a weight ratio that is from about 1:5 to about 5:1, such as from about 1:3 to about 3:1. In one particularly preferred embodiment, the composition includes a rheology modifier that is a non-ionic polymer such as a cellulose polymer and a cationic polymer (e.g., Polyquaternium-37), wherein the non-ionic polymer and the cationic polymer are present in a weight ratio that is from about 1:5 to about 5:1, such as from about 1;3 to about 3:1.

In order to improve the skin-feel of the composition, the composition may optionally include an absorbent particulate. What is meant by an absorbent particulate is a divided solid compound that can attract oil (e.g, imbibe the oil or attach the oil to its surface). Examples of absorbent particulate include, but are not limited to silica (e.g., spherical silicas, porous silicas, and fumed silica powders), Polymethyl Methacrylate, PTFE, Titanium Dioxide, Zinc Oxide, Talc, Mica, Hydroxyapatite, Magnesium Aluminometasilicate, Magnesium Aluminum Silicate, Magnesium Carbonate, Calcium Carbonate, Barium Sulphate, Tricalcium Phosphate, Silk Powder, Kaolin, Bentonite, Hectorite, and Crosslinked PMMA, Silica is particularly preferred. One notable form of silica is H53 available from Asahi Glass of Japan. Another suitable silica is Silisphere 10M commercially available from Argan of Korea.

The absorbent particulate may be present in a concentration such as from about 0.1% to about 5%. Surprisingly, rather high levels of absorbent particulate may be stabilized in compositions of the present concentration from about 0.5% to about 3%, and most preferably from about 1% to about 3%.

The composition may also include other particulates, including inorganic sunscreens such as titanium oxides or zinc oxides as long as the composition is clear, preferably transparent.

Optionally, the composition includes a silicone such as an alcohol soluble silicone fluid or silicone polymer to enhance spreading, reduce tack, and provide water resistance, without compromising the resistance to pilling of the formulation. Suitable silicone fluids include caprylyl methicone. Suitable silicone polymers include silicone elastomers. One particularly suitable silicone elastomer is a graft copolymer of an acrylic polymer backbone and dimethylpolysiloxane side chains, commercially available as a mixture of 30% copolymer and 70% cyclopentasiloxane, as KP-545, from Shin-Etsu of Japan.

Optionally, the composition may further include a photostabilizing compound that improves the stability of one or more of the organic sunscreens. Examples of photostabilizing compounds include esters of a naphthalene dicarboxylic acid, as described in U.S. Pat. No. 6.444,195 to Cole, et al., hereby incorporated by reference in its entirety. One notable photostabilizing compound is diethylhexyl 2,6 naphthalate, available as Hallbrite TQ available from Symrise GmBH of Germany.

The composition may also include cosmetically active ingredients in addition to the sunscreen, as long as the ingredients do not adversely affect the transparency and stability of the composition. What is meant by a “cosmetically active agent” is a compound (e.g., a synthetic compound or a compound isolated from a natural source) that has a cosmetic or therapeutic effect on the skin, hair, or nails, including, but not limiting to, lightening agents, darkening agents such as self-tanning agents, anti-acne agents, shine control agents, anti-microbial agents, anti-inflammatory agents, anti-mycotic agents, anti-parasite agents, external analgesics, antioxidants, keratolytic agents, detergents/surfactants, moisturizers, nutrients, vitamins, energy enhancers, anti-perspiration agents, astringents, deodorants, chemical hair removers, firming agents, anti-callous agents, and agents for hair, nail, and/or skin conditioning.

The composition may also include other functional ingredients such as humectants, chelating agents (e.g., EDTA), and preservatives (e.g., parabens), dyes, and fragrances. These other ingredients may be present in any suitable concentration known to hose skilled in the art to achieve the desired function.

In order to enhance transparency of the composition, particularly for those compositions with higher levels of organic sunscreens, the concentration of water may be restricted to low levels. In one embodiment of the invention, the concentration of water is less than about 10%, preferably less than about 5%, more preferably less than about 2%, even more preferably less than about 1%, and most preferably free of water.

In order to confer a fresh and clean sensation to the user, compositions of the present invention are “clear,” i.e., transparent or translucent, and more preferably transparent. The composition being “translucent” means that the composition has a transmittance of from about 25% to about 50%, preferably from about 35% to about 50%. In the present invention, the composition being “transparent” means that the composition has a transmittance of about 50% or more, preferably about 65% or more, more preferably about 80% or more. Most preferably about 90% or more. The transmittance are measured at 600 nm by placing the composition in a 1 cm cuvette “pathlength”, and measuring % transmission via a UV-VIS spectrophotometer, for example, UV-1601, available from Shimadzu.

Compositions of the present invention desirably have a viscosity that is from about 20,000 to about 80,000 when measured using a Brookfield viscometer, using a T spindle at 5 rpm. Surprisingly, despite the presence organic filters, typically accepted as having marginal-at-best compatibility with clear alcoholic gels, compositions of the present invention are stable to elevated temperature.

Compositions of the present invention may have a syneresis stability_{40° C.} of at least about 28 days, such as at least about 56 days, most preferably at least 84 days. In order to determine syneresis stability, 500_grams samples of the composition are placed in a plastic_package and allowed to remain in a 40° C. temperature controlled chamber (approximately 50% relative humidity). The samples are placed in the chamber 2-4 hours after making the composition and removed at 7 day intervals, allowed to equilibrate to room temperature, and observed for syneresis, i.e., a layering-type of phase separation often seen in alcohol-containing systems. If syneresis is observed, the samples is scored “0” days. If the samples passes, it is placed back in the chamber and removed at 14 days and retested. if it fails, it is scored “7 days.” If it passes, the process is repeated, scoring the sample the number of days (rounded to the nearest 7) it was in the chamber and passed syneresis evaluation. The last evaluation is at 84 days. The score is averaged for the samples for a given formulation.

Compositions of the present invention have a viscosity drop_{40° C., 28 days} of less than about 20% ,preferably less than about 15%, more preferably less than about 10%. By viscosity drop_{40° C., 28 days} it is meant the percent change in viscosity (as measured using the shear rate and spindle described above). In order to calculate the % change, the initial viscosity reading is taken 2 hours after completing the mixing of the composition. The composition is allowed to sit in the 40° C. chamber for 28 days and allowed to equilibrate to room temperature. The viscosity reading is then taken in the same manner as previously and the percent change is calculated as the absolute value of the difference in viscosities divided by the original viscosity.

Compositions of the present invention not only are clear, but also have excellent resistance to pilling. Pilling may be evaluated by evaluation by panelists who are directed to rub a given composition onto the their skin and thereafter rate the composition. One suitable test method for evaluating pilling is described in published US patent application, US 2004/0166070 to Angelike et al.

Compositions of the present invention may be used in various manners, for example, by squeezing the composition onto the hands and spreading rubbing into the skin. Alternatively, other methods are contemplated such as by spraying via pump or aerosol onto the skin, with or without subsequent rubbing.

Compositions of the present invention may be formulated to varying degrees of SPF protection. Suitable examples of compositions of the present invention may be prepared using methodology that is well known by an artisan of ordinary skill, These inventive examples as well as comparative examples are shown below:

### COMPARATIVE EXAMPLE 1

| | Ingredients | | |
|---|---|---|---|
| | Trade Name | Ingredients | % |
| 1 | | SD 40 Alcohol | 65.65 |
| 2 | Klucel | Hydroxypropylcellulose | 1.100 |
| | Hydroxypropyl- | | |
| | cellulose | | |
| 3 | Dermacryl 79 | Dermacryl 79 | 1.000 |
| 4 | | Menthol | 0.050 |
| 4 | Hallbrite TQ | Diethylhexyl 2,6-Naphthalate | 0.200 |
| 5 | | Octocrylene | 4.000 |
| 6 | | ETHYLHEXYL SALICYLATE | 5.000 |
| 7 | PARSOL 1789 | AVOBENZONE | 3.000 |
| 8 | | BENZOPHENONE -3/OXYBENZONE | 5.000 |
| 9 | | HOMOSALATE | 7.000 |
| 10 | | CAPRYLYL METHICONE | 8.000 |
| | | | 100.00 |

Comparative example 1 above was prepared. The viscosity was determined to be approximately 10,000 cps. The formulation exhibited pilling.

### COMPARATIVE EXAMPLE 2

| | Ingredients | | |
|---|---|---|---|
| | Trade Name | Ingredients | % |
| 1 | | SD 40 Alcohol (200 proof) | 60.11 |
| 2 | Synthalen CR | Polyquaternium 37 | 0.00 |
| 3 | Klucel | Hydroxypropylcellulose | 2.00 |
| | Hydroxypro- | | |
| | pylcellulose M | | |
| 4 | Hallbrite TQ | Diethylhexyl 2,6-Naphthalate | 0.700 |
| 5 | | Octocrylene | 4.000 |
| 6 | | ETHYLHEXYL SALICYLATE | 5.000 |
| 7 | PARSOL | AVOBENZONE | 3.000 |
| | 1789 | | |
| 8 | | BENZOPHENONE -3/OXYBENZONE | 6.000 |
| 9 | | HOMOSALATE | 15.000 |
| 10 | Halbrite BHB | Butyloctyl salicylate | 3.000 |
| 10 | KP-545 | Cyclopentasiloxane Acrylates | 1.000 |
| | | Dimethicone Copolymer | |
| 11 | | Menthol | 0.050 |
| 12 | Quest 36523/7 | Fragrance | 0.100 |
| 13 | | D&C Red #33 1% in alcohol | 0.0133 |
| 14 | | D&C Green # 5 1% in alcohol | 0.0266 |
| | | | 100.00 |

Comparative Example 2 was above prepared. The initial viscosity was determined to be about 44,000 cps. The formulation exhibited pilling.

### COMPARATIVE EXAMPLE 3

| | Ingredients | | |
|---|---|---|---|
| | Trade Name | Ingredients | % |
| 1 | | SD 40 Alcohol (200 proof) | 55.91 |
| 2 | Allianz OPT | Acrylates/C12-22 Alkylmethacrylate | 4.20 |
| | | Copolymer | |
| 3 | Klucel | Hydroxypropylcellulose | 2.00 |
| | Hydroxypro- | | |
| | pylcellulose M | | |
| 4 | Hallbrite TQ | Diethylhexyl 2,6-Naphthalate | 0.700 |
| 5 | | Octocrylene | 4.000 |
| 6 | | ETHYLHEXYL SALICYLATE | 5.000 |
| 7 | PARSOL | AVOBENZONE | 3.000 |
| | 1789 | | |
| 8 | | BENZOPHENONE -3/OXYBENZONE | 6.000 |
| 9 | | HOMOSALATE | 15.000 |
| 10 | Halbrite BHB | Butyloctyl salicylate | 3.000 |
| 10 | KP-545 | Cyclopentasiloxane Acrylates | 1.000 |
| | | Dimethicone Copolymer | |
| 11 | | Menthol | 0.050 |
| 12 | Quest 36523/7 | Fragrance | 0.100 |
| 13 | | D&C Red #33 1% in alcohol | 0.0133 |
| 14 | | D&C Green # 5 1% in alcohol | 0.0266 |
| | | | 100.00 |

Comparative Example 3 was above prepared. The formulation was opaque, not clear.

### COMPARATIVE EXAMPLE 4

| | Ingredients | | |
|---|---|---|---|
| | Trade Name | Ingredients | % |
| 1 | | SD 40 Alcohol (190 proof) | 60.11 |
| 2 | Synthalen CR | Polyquaternium 37 | 1.000 |
| 3 | Klucel | Hydroxypropylcellulose | 1.000 |
| | Hydroxypro- | | |
| | pylcellulose M | | |
| 4 | Hallbrite TQ | Diethylhexyl 2,6-Naphthalate | 0.700 |
| 5 | | Octocrylene | 4.000 |
| 6 | | ETHYLHEXYL SALICYLATE | 5.000 |
| 7 | PARSOL | AVOBENZONE | 3.000 |
| | 1789 | | |
| 8 | | BENZOPHENONE -3/OXYBENZONE | 6.000 |
| 9 | | HOMOSALATE | 15.000 |
| 10 | Halbrite BHB | Butyloctyl salicylate | 3.000 |
| 11 | KP-545 | Cyclopentasiloxane Acrylates | 1.000 |
| | | Dimethicone Copolymer | |
| 12 | | Menthol | 0.050 |
| 13 | Quest 36523/7 | Fragrance | 0.100 |
| 14 | | D&C Red #33 1% in alcohol | 0.0133 |
| 15 | | D&C Green # 5 1% in alcohol | 0.0266 |
| | | | 100.00 |

Comparative Example 4 was prepared. The initial viscosity was inconsistent as a poor gel network was not established. The formulation was not clear. The formulation included about 3% water.

### INVENTIVE EXAMPLE 1

| | Ingredients | | |
|---|---|---|---|
| | Trade Name | Ingredients | % |
| 1 | | SD 40 Alcohol (200 proof) | 58.21 |
| 2 | Synthalen CR | Polyquaternium 37 | 0.60 |
| 3 | Klucel | Hydroxypropylcellulose | 0.80 |
| | Hydroxypro- | | |
| | pylcellulose M | | |
| 4 | Hallbrite TQ | Diethylhexyl 2,6-Naphthalate | 0.700 |
| 5 | | Octocrylene | 4.000 |
| 6 | | ETHYLHEXYL SALICYLATE | 5.000 |
| 7 | PARSOL | AVOBENZONE | 3.000 |
| | 1789 | | |
| 8 | | BENZOPHENONE -3/OXYBENZONE | 6.000 |
| 9 | | HOMOSALATE | 15.000 |
| 10 | Halbrite BHB | Butyloctyl salicylate | 3.000 |
| 10 | KP-545 | Cyclopentasiloxane Acrylates | 1.000 |
| | | Dimethicone Copolymer | |
| 11 | Argan 10M | Silica | 2.500 |
| 12 | | Menthol | 0.050 |
| 13 | Quest 36523/7 | Fragrance | 0.100 |
| 14 | | D&C Red #33 1% in alcohol | 0.0133 |
| 15 | | D&C Green # 5 1% in alcohol | 0.0266 |
| | | | 100.00 |

Inventive Example 1 above was made by homogeneously mixing items 2 and 3 into item. Separately, items 4-11 were mixed under heat (50 C) until uniform. Mixture of items 4-11 was added to items 1-3 and mixed until uniform. Items 12-15 were each added and mixed until uniform. Q.S. with alcohol. The formulation was clear. The initial viscosity was about 38,000. The formulation did not pill.

### INVENTIVE EXAMPLE 2

| | Ingredients | | |
|---|---|---|---|
| | Trade Name | Ingredients | % |
| 1 | | SD 40 Alcohol (200 proof) | 60.11 |
| 2 | Synthalen CR | Polyquaternium 37 | 0.50 |
| 3 | Klucel | Hydroxypropylcellulose | 1.50 |
| | Hydroxypro- | | |
| | pylcellulose M | | |
| 4 | Hallbrite TQ | Diethylhexyl 2,6-Naphthalate | 0.700 |
| 5 | | Octocrylene | 4.000 |
| 6 | | ETHYLHEXYL SALICYLATE | 5.000 |
| 7 | PARSOL | AVOBENZONE | 3.000 |
| | 1789 | | |
| 8 | | BENZOPHENONE -3/OXYBENZONE | 6.000 |
| 9 | | HOMOSALATE | 15.000 |
| 10 | Halbrite BHB | Butyloctyl salicylate | 3.000 |
| 10 | KP-545 | Cyclopentasiloxane Acrylates | 1.000 |
| | | Dimethicone Copolymer | |
| 11 | | Menthol | 0.050 |
| 12 | Quest 36523/7 | Fragrance | 0.100 |
| 13 | | D&C Red #33 1% in alcohol | 0.0133 |
| 14 | | D&C Green # 5 1% in alcohol | 0.0266 |
| | | | 100.00 |

Inventive Example 2 above was made by homogeneously mixing items 2 and 3 into item. Separately, items 4-11 were mixed under heat (50 C) until uniform. Mixture of items 4-10 was added to items 1-3 and mixed until uniform. Items 11-14 were each added and mixed until uniform. Q.S. with alcohol. The formulation was clear. The initial viscosity was about 52,000. The formulation did not pill.

### INVENTIVE EXAMPLE 3

| | Ingredients | | |
|---|---|---|---|
| | Trade Name | Ingredients | % |
| 1 | | SD 40 Alcohol (200 proof) | 60.11 |
| 2 | Synthalen CR | Polyquaternium 37 | 1.50 |
| 3 | Klucel | Hydroxypropylcellulose | 0.50 |
| | Hydroxypro- | | |
| | pylcellulose M | | |
| 4 | Hallbrite TQ | Diethylhexyl 2,6-Naphthalate | 0.700 |
| 5 | | Octocrylene | 4.000 |
| 6 | | ETHYLHEXYL SALICYLATE | 5.000 |
| 7 | PARSOL | AVOBENZONE | 3.000 |
| | 1789 | | |
| 8 | | BENZOPHENONE -3/OXYBENZONE | 6.000 |
| 9 | | HOMOSALATE | 15.000 |
| 10 | Halbrite BHB | Butyloctyl salicylate | 3.000 |
| 10 | KP-545 | Cyclopentasiloxane Acrylates | 1.000 |
| | | Dimethicone Copolymer | |
| 11 | | Menthol | 0.050 |
| 12 | Quest 36523/7 | Fragrance | 0.100 |
| 13 | | D&C Red #33 1% in alcohol | 0.0133 |
| 14 | | D&C Green # 5 1% in alcohol | 0.0266 |
| | | | 100.00 |

Inventive Example 3 above was made by homogeneously mixing items 2 and 3 into item. Separately, items 4-10 were mixed under heat (SOC) until uniform. Mixture of items 4-10 was added to items 1-3 and mixed until uniform. Items 11-14 were each added and mixed until uniform. Q.S. with alcohol. The formulation was clear. The initial viscosity was about 35,000. The formulation did not pill.

What is claimed is:

1. A personal care composition comprising an organic sunscreen, an alcohol, a rheology modifier comprising a cellulose polymer, and an anti-pilling agent comprising a cationic polymer.

2. The composition of claim 1 further comprising an absorbent particulate.

3. The composition of claim 1, wherein the cellulose polymer and the cationic polymer are soluble in the alcohol.

4. The composition of claim 1 comprising less than about 10% water.

5. The composition of claim 1, wherein the weight ratio of the cellulose polymer to the cationic polymer is from about 1:5 to about 5:1.

6. The composition of claim 1 having % transmittance_{600 nm} greater than about 25%.

7. The composition of claim 1 having a syneresis stability_{40° C.} of at least about 28 days.

8. The composition of claim 1 having a viscosity drop 40° C., 28 days of less than about 20%.

9. A personal care composition comprising an organic sunscreen and an alcohol, wherein the compositions has a % transmittance_{600 nm} greater than about 25% and a syneresis stability_{40° C.} of at least about 28 days.

10. A personal care composition comprising an organic sunscreen and an alcohol, wherein the compositions has a % transmittance_{600 nm} greater than about 25% and a viscosity drop 40° C., 28 days of less than about 20%.
